(19) United States
(12) Patent Application Publication
(10) Pub. No.: US 2014/0127148 A1
(43) Pub. Date: May 8, 2014

(54) FLUID COMPOSITION CONTAINING A SUN FILTER, AND USE THEREOF FOR THE TREATMENT OF SCARS

(71) Applicant: Nathalie Derain, Prenois (FR)

(72) Inventor: Nathalie Derain, Prenois (FR)

(73) Assignee: LABORATOIRES URGO, Chenove (FR)

(21) Appl. No.: 14/007,558

(22) PCT Filed: Mar. 23, 2012
(86) PCT No.: PCT/FR2012/050603

§ 371 Date: Jan. 3, 2014

(30) Foreign Application Priority Data
Mar. 25, 2011 (FR) 1152497

(51) Int. Cl. A61K 8/37; A61K 8/35; A61Q 17/04

(52) U.S. Cl. CPC A61K 8/37; A61Q 17/04; A61K 8/35
USPC 424/59

(57) ABSTRACT

The invention relates to a fluid composition containing at least a polymer, a plasticiser, a volatile solvent, and a very small amount of an organic sun filter in the order of between 0.5 and 2.2 wt.-% in relation to the total weight of the composition. The composition, which is intended to be applied to the skin, can be used to treat scars. In addition, the composition advantageously contains a derivative of dibenzoylmethane and a cinnamate derivative.

The present invention relates to a liquid dressing containing at least a polymer, a plasticizer, a volatile solvent, and an organic sun filter in very small amount. This composition, intended to be applied on the skin to cover it with a waterproof film, can be used advantageously for treating scars.

Scars are particularly fragile and sensitive not only to external pathogens, but also to ultraviolet (UV) rays from the sun.

Several products have already been proposed commercially for protecting scars, notably with respect to UV radiation. Some of them are in the form of emulsion creams and contain a large amount of water. These water-based products comprise very high contents of sun filters to guarantee adequate efficacy against UV radiation (of the order of 10 to 35 wt %).

A scar-reducing cream containing from 2 to 7.5 wt % of octinoxate and from 0.5 to 7.5 wt % of zinc oxide was described in document U.S. Pat. No. 7,241,451. However, this cream has the drawback of poor water resistance, so that the sun protection of the scar disappears once the skin comes in contact with water. Moreover, this type of cream has the drawback that it is not resistant to rubbing.

Furthermore, a certain proportion of the sun filters that these formulations contain tend to penetrate into the skin, so that to guarantee effective protection of the skin against UV radiation, the composition must contain a high proportion of organic filter(s).

Liquid dressings formulated in a volatile solvent are generally in the form of a fluid that is applied on the skin using a suitable applicator, such as a spray, a brush, a spatula or similar. The volatile solvent evaporates in contact with the skin so as to leave a solid film which protects the skin.

Liquid dressings forming a film on the skin offer the advantage of limiting the penetration of the constituents into the skin, since the film formed is solid and remains on the surface. Moreover, as these films are resistant to water and to rubbing, they may last for several days after application.

A liquid dressing in solvent phase containing large amounts of organic sun filters and inorganic sun filters was proposed in document FR 2 929 522. However, the applicant established that the inorganic filters settle over time, so that the composition is not stable in storage.

Moreover, in contrast to the teaching of this document, the applicant has demonstrated that it is not necessary to incorporate an inorganic sun filter to obtain effective UV protection. The applicant has thus optimized the nature and amount of sun filter to be incorporated in a liquid dressing in solvent phase.

Furthermore, the applicant discovered, against all expectation, that the organic filter butyl methoxydibenzoylmethane (avobenzone) still has, in this type of formulation, high sun protection capacity even after irradiation, so that it is not necessary to add to it a photostabilizer, such as octocrylene that is used conventionally in combination with the latter.

The present invention thus proposes liquid dressings offering satisfactory absorption in the UV using a limited amount of sun filter(s), preferably organic.

The present invention therefore relates to a fluid composition intended to be applied on the skin containing a polymer, a plasticizer, a solvent of said polymer apart from water which is volatile, and at least one organic sun filter, characterized in that the total content of sun filter(s) is between 0.5 and 2.2 wt % of the total weight of the composition, the volatile solvent represents from 60 to 90 wt % relative to the total weight of the composition, and the polymer represents from 5 to 20% in dry weight of the total weight of the composition.

The polymer is preferably able to form a continuous film on the skin, after application of the composition according to the invention. The polymer is preferably water-insoluble and forms a water-insoluble film once the volatile solvent of the composition has evaporated. For example, hydroxypropylcellulose and hydroxyethylcellulose do not form water-insoluble dry films. The polymer is preferably soluble in a volatile solvent other than water.

The polymer can be selected from the polymers of natural origin, guar gums, gums arabic, alginates, xanthan gum, gelatin, chitosan, silicates, hydrated silicas, polyurethanes, polyesters, polyester amides, polyamides, polyureas, vinyl polymers, acrylic polymers, polyvinyl butyrals, alkyd resins, resins from aldehyde condensation products such as arylsulfonamide formaldehyde resins, arylsulfonamide epoxy resins, ethyltosylamide resins, acetophenone/formaldehyde resins, and mixtures thereof.

The vinyl polymers can result from the homopolymerization or copolymerization of monomers selected from the vinyl esters such as vinyl acetate, vinyl neodecanoate, vinyl pivalate, vinyl benzoate and t-butyl vinyl benzoate and styrene monomers such as styrene and alpha-methylstyrene.

The acrylic polymers can be homopolymers of acrylic acid, copolymers of acrylic acid, homopolymers of methacrylic acid or copolymers of methacrylic acid.

According to one embodiment, the polymer is selected from the cellulosic polymers notably hydroxypropylmethylcellulose, the cellulose ethers, the cellulose esters, the nitrocelluloses, the water-insoluble cellulose derivatives, and mixtures thereof.

According to one embodiment, the polymer is a nitrocellulose, preferably selected from the nitrocelluloses of high viscosity. It is notably possible to use nitrocelluloses of grade between RS ½ second and RS 20 seconds according to the US standard, corresponding to a grade between 8E and 21E according to the European standard. For example, nitrocellulose of grade 10E or 11E (RS 15 seconds according to the US standard) is preferred.

The nitrocellulose can notably be selected from the nitrocelluloses RS 5 sec. and RS 15 sec. marketed by the company HERCULES, the products DHL® 120/170, DHL® 25/45 or DHX® 40/70 marketed by Nobel Enterprises, the nitrocelluloses E 840® and E 620® produced by Wolff Cellulosics, and the products marketed under references E80®, E70®, E60® and E40® by SNPE-Bergerac. The nitrocellulose can be supplied in dry form or in solution in a solvent such as isopropanol or ethanol.

The polymer is preferably present in a content in the range from 5 to 20 dry wt % of the total weight of the composition, for example from 6 to 15% or even from 6 to 9 dry wt %, relative to the total weight of the composition.

The plasticizer can make the film more flexible; it is advantageously selected from oils of vegetable origin and ethoxylated derivatives thereof. The vegetable oil can be:

The vegetable oil is advantageously selected from sesame oil, castor oil, almond oil, canola oil, hazelnut oil, pistachio oil, linseed oil, borage oil, hemp oil, jojoba oil, sunflower oil, wheat-germ oil, maize oil and/or maize-germ oil, peanut oil, avocado oil, safflower oil, colza oil, olive oil, argan oil, sunflower oil, grapeseed oil, soybean oil, walnut oil, pumpkin seed oil, palm oil, copra oil, and mixtures thereof.

The oil can also be a derivative of one of the aforementioned vegetable oils. It can be hydrogenated or non-hydrogenated, peroxidized or non-peroxidized oil.

The plasticizer can also be selected from:

monocarboxylic acid esters such as isononyl isononanoate, oleyl erucate or octyl-2-docecyl neopentanoate;

fatty alcohols such as octyldodecanol, 2-butyloctanol, 2-hexyldecanol, 2-undecylpentadecanol, oleic alcohol;

fatty acids such as oleic acid, linoleic acid, linolenic acid;

glycols and derivatives thereof, such as diethylene glycol ethyl ether, diethylene glycol methyl ether, polyethylene glycols, polypropylene glycols;

dicarboxylic acid esters, such as citrates (acetyltributyl citrate), phthalates (dibutyl phthalate), adipates, sebacates (dibutyl sebacate);

triacetin or glyceryl triacetate;

glycerin;

mixtures thereof.

The plasticizer is preferably soluble in the solvent of the polymer.

Advantageously, the plasticizer and the polymer are present in the composition according to the invention in a weight ratio in the range from 0.5 to 1.5, and preferably in the range from 0.7 to 1.2, more particularly close to 1.

In the context of the present invention, organic sun filter means any organic compound absorbing UV radiation in the wavelength range generally from 280 nm to 400 nm. The organic sun filter used according to the invention can be a compound filtering UV-A radiation with wavelengths between 320 and 400 nm, a compound filtering UV-B radiation with wavelengths between 280 and 320 nm, a compound filtering UV-A and UV-B radiation or a mixture thereof.

The composition preferably comprises less than 0.5 wt %, more preferably less than 0.2 wt % of inorganic sun filter(s) relative to the total weight of the composition.

The composition of the invention is preferably free from inorganic sun filter.

The total amount of sun filter(s) in the composition is low, preferably between 0.5 and 2.2%, more preferably between 1.2 and 1.8%, preferably of the order of 1.5 wt % relative to the total weight of the composition.

The organic filter can be selected from organic filters active in the UV-A, organic filters active in the UV-B, organic filters active in the UV-A and UV-B, and mixtures thereof.

An organic filter active in the UV-A is advantageously selected from:

the derivatives of dibenzoylmethane, for example butyl methoxydibenzoylmethane (INCI name) or Avobenzone (USAN name) notably sold under the trade name PARSOL® 1789, or isopropyl dibenzoylmethane,

menthyl anthranilate notably sold under the reference NEO HELIOPAN® MA by SYMRISE,

mixtures thereof.

The composition of the invention advantageously contains a derivative of dibenzoylmethane without admixture of a photostabilizer, such as octocrylene. According to one embodiment, a fat-soluble organic filter absorbing in the UV-B is added to the dibenzoylmethane derivative.

The content of organic filter active in the UV-A, in particular of dibenzoylmethane derivative, is advantageously below 1 wt %, for example between 0.2 and 0.8 wt %, preferably between 0.3 and 0.7 wt % and more preferably of the order of 0.5 wt % relative to the weight of the composition.

The composition can contain a filter active in the UV-B, preferably fat-soluble, which can be selected from sun filters absorbing only wavelengths located in the UV-B, sun filters absorbing both in the UV-A and UV-B, and mixtures thereof.

The filter active only in the UV-B can be selected from:

the salicylic derivatives: homosalate notably sold under the name NEO HELIOPAN® HMS; ethylhexyl salicylate notably sold under the name NEO HELIOPAN® OS by SYMRISE; octyl salicylate notably sold under the name NEO HELIOPAN® type 05;

the cinnamates: ethylhexyl methoxycinnamate notably sold under the trade name PARSOL® MCX by DSM NutritionalProducts, Inc.; isopropyl methoxycinnamate; isoamyl methoxycinnamate notably sold under the trade name NEO HELIOPAN® E 1000 by SYMRISE; diisopropyl methylcinnamate; cinnoxate; glyceryl ethylhexanoate-dimethoxycinnamate;

the benzylidene camphor derivatives: 3-benzylidene camphor manufactured under the name MEXORYL® SD by CHIMEX; methylbenzylidene camphor notably sold under the name NEO HELIOPAN® MBC;

the triazine derivatives: ethylhexyltriazone notably sold under the trade name UVINUL® T150 by BASF, diethylhexyl butamidotriazone sold under the trade name UVASORB® HEB, bis-ethylhexyloxyphenolmethoxyphenyltriazine;

the para-aminobenzoates, such as ethyl PABA; ethyl dihydroxypropyl PABA; ethylhexyldimethyl PABA (ESCALOL® 507 from ISP);

the imidazoline derivatives: ethylhexyldimethoxybenzylidene dioxoimidazoline propionate;

the derivatives of benzal malonate: polyorganosiloxanes with benzal malonate function such as Polysilicone-15 notably sold under the trade name PARSOL® SLX by DSM Nutritional Products Inc.; dineopentyl 4′-methoxybenzalmalonate;

and mixtures thereof.

A filter active both in the UV-A and in the UV-B can be selected from:

the benzophenone derivatives: benzophenone-1 sold under the trade name UVINUL® 400; benzophenone-2 sold under the trade name UVINUL D50; benzophenone-3 or oxybenzone sold under the trade name UVINUL® M40; benzophenone-4 sold under the trade name UVINUL® MS40; benzophenone-6 sold under the trade name HELISORB 11; benzophenone-8 sold under the trade name SPECTRASORB® UV-24;

the phenylbenzotriazole derivatives: drometrizole trisiloxane notably sold under the name Silatrizole® by RHODIA CHIMIE; methylene bis-benzotriazolyl tetramethylbutylphenol sold in solid form notably under the trade name MIXXIM® BB/100 by FAIRMOUNT CHEMICAL;

the bis-resorcinyltriazine derivatives: bis-ethylhexyloxyphenolmethoxyphenyltriazine notably sold under the trade name TINOSORB® S by CIBA GEIGY;

the benzoxazole derivatives: 2,4-bis-[5-1(dimethylpropyl)benzoxazol-2-yl-(4-phenyl)-imino]-6-(2-ethylhexyl)-imino-1,3,5-triazine notably sold under the name Uvasorb® K2A by Sigma 3V;

and mixtures thereof.

According to one embodiment of the invention the organic sun filter is a mixture of a sun filter absorbing in the UV-A and a sun filter filtering in the UV-B.

For example, a fat-soluble UV-B filter selected from the cinnamates, notably ethylhexyl methoxycinnamate (INCI name) or octinoxate (USAN name) sold under the trade name PARSOL® MCX can be added to an organic filter active in the UV-A.

The organic sun filter can be a mixture of butyl methoxydibenzoylmethane (USAN avobenzone) and ethylhexyl methoxycinnamate (USAN octinoxate).

The content of fat-soluble organic UV-B filter, notably of ethylhexyl methoxycinnamate, in the composition of the invention is advantageously below 1.2%, for example between 0.5 and 1.2 wt %, preferably between 0.8 and 1.2 wt %, more preferably of the order of 1 wt % relative to the weight of the composition.

According to one embodiment, the weight ratio of the derivative of dibenzoylmethane to the cinnamate derivative is between 0.3 and 0.7, preferably of the order of 0.5.

The organic sun filter comprising a dibenzoylmethane derivative and a cinnamate derivative is advantageously present in an amount between 0.5 and 2.2 wt %, preferably between 1.2 and 1.8 wt %, more preferably of the order of 1.5 wt % relative to the total weight of the composition.

The compositions of the invention also contain a volatile solvent, which preferably dissolves the polymer and the plasticizer. This makes it possible to dissolve and homogenize the ingredients and its volatile character helps to form a continuous solid film on the skin following application.

In the context of the present invention, volatile solvent means a solvent capable of evaporating rapidly in contact with the skin. Water is excluded from this definition. Volatile solvents or mixtures of volatile solvents are preferred whose boiling point is above 50° C. (at atmospheric pressure).

As solvent that can be used, we may mention:

ketones such as methyl ethyl ketone, methyl isobutyl ketone, diisobutyl ketone, isophorone, cyclohexanone, acetone;

alcohols such as ethanol, isopropanol, n-propanol, n-butanol, diacetone alcohol, 2-butoxyethanol, cyclohexanol;

glycols such as ethylene glycol, propylene glycol, pentylene glycol, glycerol;

propylene glycol ethers such as propylene glycol monomethyl ether, propylene glycol monomethyl ether acetate, dipropylene glycol mono-n-butyl ether;

esters such as ethyl acetate, methyl acetate, propyl acetate, n-butyl acetate, isopentyl acetate;

methylal;

ethers such as diethyl ether, dimethyl ether or dichlorodiethyl ether;

the silicone solvents, notably hexamethylsiloxane and polydimethylsiloxanes;

mixtures thereof.

The volatile solvent will advantageously be selected from ethanol, ethyl acetate and mixtures thereof. In particular, a mixture of ethyl acetate and ethanol in proportions between 1/1 and 3/1, preferably 2/1, will be selected. The amount of volatile solvent preferably represents from 60 to 90 wt % relative to the total weight of the composition, advantageously from 70 to 90 wt % relative to the total weight of the composition.

The composition is essentially water-free. It preferably contains less than 2 wt % of water.

The compositions according to the invention can also contain additives usually employed in the preparation of dressings, such as perfumes, preservatives, depigmenting agents, antibacterial agents, antifungal agents, healing agents, analgesics, anti-inflammatories, hydrating agents, keratolytic agents, vitamins, glycerin, citric acid.

In general, the active agents are selected from:

antibacterials such as Polymyxin B, the penicillins (amoxicillin), clavulanic acid, the tetracyclines, minocycline, chlortetracycline, the aminoglycosides, amikacin, gentamicin, neomycin, silver and silver salts (sulfadiazine silver), probiotics;

antiseptics such as sodium mercurothiolate, eosin, chlorhexidine, phenylmercuric borate, hydrogen peroxide, Dakin solution, triclosan, biguanide, hexamidine, thymol, Lugol, iodinated povidone, merbromin, benzalkonium chloride and benzethonium chloride, ethanol, isopropanol;

antivirals such as acyclovir, famciclovir, ritonavir;

antifungals such as the polyenes, nystatin, amphotericin B, natamycin, the imidazoles (miconazole, ketoconazole, clotrimazole, econazole, bifonazole, butoconazole, fenticonazole, isoconazole, oxiconazole, sertaconazole, sulconazole, thiabendazole, tioconazole), the triazoles (fluconazole, itraconazole, ravuconazole, posaconazole, voriconazole), the allylamines, terbinafine, amorolfine, naftifine, butenafine;

flucytosine (antimetabolite), griseofulvin, caspofungin, micafungin;

analgesics such as paracetamol, codeine, dextropropoxyphene, tramadol, morphine and its derivatives, the corticoids and derivatives;

anti-inflammatories such as the glucocorticoids, the nonsteroidal anti-inflammatories, aspirin, ibuprofen, ketoprofen, flurbiprofen, diclofenac, aceclofenac, ketorolac, meloxicam, piroxicam, tenoxicam, naproxen, indometacin, naproxcinod, nimesulide, celecoxib, etoricoxib, parecoxib, rofecoxib, valdecoxib, phenylbutazone, niflumic acid, mefenamic acid;

active agents that promote healing such as retinol, vitamin A, vitamin E, N-acetylhydroxyproline, extracts of Centella asiatica, papain, the silicones, the essential oils of thyme, of niaouli, of rosemary and of sage, hyaluronic acid, the synthetic polysulfated oligosaccharides having 1 to 4 monosaccharide units such as the potassium salt of sucrose octasulfate, the silver salt of sucrose octasulfate or sucralfate, allantoin;
depigmenting agents such as kojic acid (Kojic Acid SL®—Quimasso (Sino Lion)), arbutin (Olevatin®—Quimasso (Sino Lion)), the mixture of sodium palmitoylpropyl and extract of white water lily (Sepicalm®—Seppic), undecylenoyl phenylalanine (Sepiwhite®—Seppic), licorice extract obtained by fermentation of Aspergillus and ethoxydiglycol (Gatuline Whitening®—Gattefosse), octadecenedioic acid (ODA White®—Sederma), alpha-arbutin (Alpha-Arbutin®, SACI-CFPA (Pentapharm)), aqueous extract of Arctophylos Uva Ursi leaves (Melfade-J®—SACI-CFPA (Pentapharm)), the complex plant mixture Gigawhite® (SACI-CFPA (Alpaflor)), diacetyl boldine (Lumiskin®—Sederma), extract of Japanese mandarin (Melaslow®—Sederma), the mixture of lemon extract enriched with citric acid and cucumber extract (Uninontan®U-34—Unipex), the mixture of extract of Rumex occidentalis and of vitamin C (Tyrostat® 11—Unipex), oligopeptides (Melanostatin 5®—Unipex), kojic dipalmitate (KAD-15®—Quimasso (Sino Lion)), the complex of natural origin Vegewhite® from LCW, wheat-germ extracts (Clariskin® II—Silab), ethyldiamine triacetate (EDTA);

keratolytic agents such as salicylic acid, zinc salicylate, ascorbic acid, the alpha hydroxylated acids (glycolic, lactic, malic, citric, tartaric acid), the extracts of silver maple, of morello cherry tree, of tamarind, urea, the topical retinoid Kératoline® (Sederma), the proteases obtained by fermentation of Bacillus subtilis, the product Linked-Papain® (SACI-CFPA), papain (proteolytic enzyme from the papaya fruit);

restructuring active agents (for example agents for restructuring the appendages) such as silica derivatives, vitamin E, chamomile, calcium, horsetail extract, Lipester de Soie;

anesthetics such as benzocaine, lidocaine, dibucaine, pramoxine hydrochloride, bupivacaine, mepivacaine, prilocaine, etidocaine.

As already mentioned, the composition according to the present invention is in the form of a fluid liquid intended to be applied using a suitable device. It is advantageously formulated so that it can be used in various possible forms, notably in the form of a bottle with suitable applicator, such as a brush or a spatula, a roll-on, or in the form of spray, lipstick, or mousses.

The invention further relates to the composition described above for use in the protection of scars.

According to one embodiment, the compositions according to the present invention are intended to be applied on scars, whether they are connected with an accident, a disease or following surgery.

These compositions can also be used for any skin disorder for which UV exposure is undesirable.

As examples, we may mention acne, chickenpox, shingles, rosacea, first-degree burns, eczema, hyperpigmentation, actinic dermatitis, vitiligo, xerosis, porphyria, stretchmarks, psoriasis, insect bites.

The invention also relates to the use of a composition as defined above for preparing a dressing intended for protecting areas at risk, such as scars.

The following examples illustrate the invention, without limiting its scope.

### EXAMPLE 1

The following composition was prepared.

| | Manufacturer/ | | | |
|---|---|---|---|---|
| Trade name | Supplier | INCI name | CAS No. | wt % |
| 1. DHL ® | Nobel | Nitrocellulose | 9004-70-0 | 11.5 |
| 120/170 IPA | | | | |
| 2. Castor oil | Prod'hyg | Ricinus Communis | 8001-79-4 | 10.0 |
| | Laboratoire | (Castor) Seed Oil | | |
| 3. Absolute | Charbonneau | Alcohol | 64-17-5 | 25.7 |
| ethanol | Brabant | | | |
| 4. Ethyl acetate | Darfeuille | Ethyl acetate | 141-78-6 | 51.3 |
| 5. Parsol ® 1789 | DSM | Butyl methoxy- | 70356-09-1 | 0.5 |
| | | dibenzoylmethane | | |
| 6. Parsol ® MCX | DSM | Ethylhexyl | 5466-77-3 | 1.0 |
| | | methoxycinnamate | | |

### Procedure:

Ingredients 2 to 6 were mixed with stirring at 700 rev/min for 10 minutes with a propeller stirrer. The nitrocellulose was then dispersed in the mixture while stirring, and the mixture was stirred for 20 minutes at 1100 rev/min and then for 30 minutes at 2000 rev/min.

### Measurement of Filtering Capacity:

The composition according to the invention was spread uniformly and in a metered amount on an inert substrate, then it was left to dry so that the volatile solvent evaporated. A film was obtained with a thickness of 10 microns, and was separated from the substrate.

The sun protection factor (SPF) of the composition was measured in vitro on the film of composition previously irradiated so as to approach the conditions encountered in vivo. The film was irradiated beforehand for 30 minutes with a dose of standard sunlight between 2 and 4 MED. The MED value adopted was 25 mJ/cm² erythema efficacy, this value corresponding to exposure for 15 minutes to the sun at its zenith (standard sun) with an erythema power of 4 MED/h, the UVB spectroradiometric flux being 0.365 mW/cm² between 290 and 320 nm, and the UVA spectroradiometric flux being 6.0 mW/cm² between 320 and 400 nm (UVA/UVB ratio: 16.5/1).

The method used for measuring the SPF employed the principles described by B. L. Diffey and J. Robson in J.S.C.C., 40, 127-133, May-June 1989.

The equipment used comprised:

A “KONTRON 930” spectrophotometer equipped with a UV source and a double monochromator capable of delivering an energy flux between 250 and 800 nm;

A luminous source for the irradiation: CPS+SUNTEST (ATLAS) with standard filter and Schott WG 320 filter. A sheet of PMMA containing UV filters is used as reference control.

The mean value of the SPF obtained from 3 to 5 measurements on the 10 μm film was 243 (±74).

The film of the invention has protective properties with respect to UVA and UVB, with a good spectral distribution between UVA and UVB. The critical wavelength is 370 nm.

### EXAMPLE 2

The following composition was prepared following the procedure in example 1.

| | Manufacturer/ | | | |
|---|---|---|---|---|
| Trade name | Supplier | INCI name | CAS No. | wt % |
| 1. DHL ® | Nobel | Nitrocellulose | 9004-70-0 | 11.5 |
| 120/170 IPA | | | | |
| 2. Castor oil | Prod'hyg | Ricinus Communis | 8001-79-4 | 10.0 |
| | Laboratoire | (Castor) Seed Oil | | |
| 3. Absolute | Charbonneau | Alcohol | 64-17-5 | 26.0 |
| ethanol | Brabant | | | |
| 4. Ethyl acetate | Darfeuille | Ethyl acetate | 141-78-6 | 52.0 |
| 5. Parsol ® MCX | DSM | Ethylhexyl | 5466-77-3 | 0.5 |
| | | methoxycinnamate | | |

The mean value of the SPF was calculated according to the method in example 1. The mean value of the SPF obtained after pre-irradiation from 5 measurements was 17.10 (±0.32). The product therefore did indeed provide effective sun protection.

### COMPARATIVE EXAMPLE 3

The following composition was prepared:

| | Manufacturer/ | | | |
|---|---|---|---|---|
| Trade name | Supplier | INCI name | CAS No. | wt % |
| 1. DHL ® | Nobel | Nitrocellulose | 9004-70-0 | 25.0 |
| 120/170 IPA | | | | |
| 2. Castor oil | Prod'hyg | Ricinus Communis | 8001-79-4 | 21.7 |
| | Laboratoire | (Castor) Seed Oil | | |
| 3. Absolute | Charbonneau | Alcohol | 64-17-5 | 17.3 |
| ethanol | Brabant | | | |
| 4. Ethyl acetate | Darfeuille | Ethyl acetate | 141-78-6 | 34.5 |
| 5. Parsol ® 1789 | DSM | Butyl methoxy- | 70356-09-1 | 0.5 |
| | | dibenzoylmethane | | |
| 6. Parsol ® MCX | DSM | Ethylhexyl | 5466-77-3 | 1.0 |
| | | methoxycinnamate | | |

### Procedure:

Ingredients 2 to 6 were mixed with stirring at 700 rev/min for 10 minutes with a propeller stirrer. The nitrocellulose was then dispersed in the mixture while stirring, and the mixture was stirred for 20 minutes at 1100 rev/min and then for 30 minutes at 2000 rev/min.

The content of nitrocellulose (percentage by weight dry) in example 4 was 20%. The composition according to example 4 was very viscous and thready. Accordingly, it was impossible to spread it with a brush. The film obtained was very irregular and nonhomogeneous.

### COMPARATIVE EXAMPLE 4

The following composition was prepared:

| | Manufacturer/ | | | |
|---|---|---|---|---|
| Trade name | Supplier | INCI name | CAS No. | wt % |
| 1. DHL ® | Nobel | Nitrocellulose | 9004-70-0 | 6.0 |
| 120/170 IPA | | | | |
| 2. Castor oil | Prod'hyg | Ricinus Communis | 8001-79-4 | 5.2 |
| | Laboratoire | (Castor) Seed Oil | | |
| 3. Absolute | Charbonneau | Alcohol | 64-17-5 | 29.1 |
| ethanol | Brabant | | | |
| 4. Ethyl acetate | Darfeuille | Ethyl acetate | 141-78-6 | 58.2 |
| 5. Parsol ® 1789 | DSM | Butyl methoxy- | 70356-09-1 | 0.5 |
| | | dibenzoylmethane | | |
| 6. Parsol ® MCX | DSM | Ethylhexyl | 5466-77-3 | 1.0 |
| | | methoxycinnamate | | |

### Procedure:

Ingredients 2 to 6 were mixed with stirring at 700 rev/min for 10 minutes with a propeller stirrer. The nitrocellulose was then dispersed in the mixture while stirring, and the mixture was stirred for 20 minutes at 1100 rev/min and then for 30 minutes at 2000 rev/min.

The content of nitrocellulose (percentage by weight dry) in example 5 was 4.8%. The composition according to example 5 was very liquid and did not allow a thick enough film to be obtained on the skin to give the required sun protection. The film formed was extremely thin, making it more sensitive to rubbing and washing.

### COMPARATIVE EXAMPLE 5

The following composition was prepared:

| | Manufacturer/ | | | |
|---|---|---|---|---|
| Trade name | Supplier | INCI name | CAS No. | wt % |
| 1. DHL ® | Nobel | Nitrocellulose | 9004-70-0 | 11.5 |
| 120/170 IPA | | | | |
| 2. Castor oil | Prod'hyg | Ricinus Communis | 8001-79-4 | 10.0 |
| | Laboratoire | (Castor) Seed Oil | | |
| 3. Absolute | Charbonneau | Alcohol | 64-17-5 | 25.4 |
| ethanol | Brabant | | | |
| 4. Ethyl acetate | Darfeuille | Ethyl acetate | 141-78-6 | 50.9 |
| 5. Parsol ® 1789 | DSM | Butyl methoxy- | 70356-09-1 | 1.0 |
| | | dibenzoylmethane | | |
| 6. Parsol ® MCX | DSM | Ethylhexyl | 5466-77-3 | 1.2 |
| | | methoxycinnamate | | |

### Procedure:

Ingredients 2 to 6 were mixed with stirring at 700 rev/min for 10 minutes with a propeller stirrer. The nitrocellulose was then dispersed in the mixture while stirring, and the mixture was stirred for 20 minutes at 1100 rev/min and then for 30 minutes at 2000 rev/min.

### Measurement of Filtering Capacity:

The composition according to example 6 was spread uniformly and in a metered amount on an inert substrate, then it was left to dry so that the volatile solvent evaporated. A film was obtained with a thickness of 10 microns, and was separated from the substrate.

The sun protection factor (SPF) of the composition was measured as in example 1.

The spectrophotometer showed saturation above 1.2 wt % of Parsol MCX. Moreover, for Parsol 1789, saturation was shown at 1 wt %.

1. A fluid composition intended to be applied on the skin containing a polymer, a plasticizer, a solvent of said polymer apart from water which is volatile, and at least one organic sun filter, wherein the total content of sun filter(s) is between 0.5 and 2.2 wt % of the total weight of the composition, the volatile solvent represents from 60 to 90 wt % relative to the total weight of the composition, and the polymer represents from 5 to 20 dry wt % of the total weight of the composition.

2. The composition as claimed in claim 1, wherein the total content of sun filter(s) is between 1.2 and 1.8 wt % of the total weight of the composition, preferably of the order of 1.5 wt %.

3. The composition as claimed in claim 1, wherein the organic sun filter is a mixture of a dibenzoylmethane derivative and a fat-soluble organic filter absorbing in the UV-B.

4. The composition as claimed in claim 3, wherein the dibenzoylmethane derivative represents between 0.3 and 0.7 wt % of the total weight of the composition.

5. The composition as claimed in claim 3, wherein the fat-soluble organic filter absorbing in the UV-B represents from 0.8 to 1.2 wt % of the total weight of the composition.

6. The composition as claimed in claim 3, wherein the organic sun filter is a mixture of butyl methoxydibenzoylmethane (USAN avobenzone) and ethylhexyl methoxycinnamate (USAN octinoxate).

7. The composition as claimed in claim 1, which contains less than 0.5%, preferably less than 0.2 wt % of inorganic sun filter(s) relative to the total weight of the composition.

8. The composition as claimed in claim 1, wherein the polymer is a nitrocellulose.

9. The composition as claimed in claim 1, wherein the plasticizer is selected from castor oil, wheat-germ oil, dibutyl phthalate, glycerin, dibutyl sebacate, acetyltributyl citrate, triacetin, and mixtures thereof.

10. The composition as claimed claim 1, wherein the solvent of said polymer is selected from ethanol, ethyl acetate and mixtures thereof.

11. The composition as claimed in claim 1, which further comprises an agent that promotes healing, an antibacterial agent, an antifungal agent, a depigmenting agent, an analgesic, an anti-inflammatory, a hydrating agent, a keratolytic agent, vitamins, or a mixture of these compounds.

12. The composition as claimed in claim 1 for use as a dressing for protecting scars.
